(19) United States
(12) Patent Application Publication
(10) Pub. No.: US 2006/0041188 A1
(43) Pub. Date: Feb. 23, 2006

(54) Flexible endoscope

(76) Inventors: Carlo A Dirusso, Bronx, NY (US); Edward Grabover, Danbury, CT (US); Gregory S Konstorum, Stamford, CT (US)

Correspondence Address: HARRINGTON & SMITH, LLP, 4 RESEARCH DRIVE, SHELTON, CT, 06484-6212, US

(21) Appl. No.: 10/518,222

(22) PCT Filed: Mar. 25, 2003
(86) PCT No.: PCT/US03/09155

§ 371 Date: Dec. 15, 2004

(51) Int. Cl. A61B 1/00

(52) U.S. Cl. 600/146; 600/152; 600/139

(57) ABSTRACT

An endoscope including a handle; and a shaft extending from the handle. The shaft has a front end with a first active deflection section and a second active deflection section. The first active deflection section is limited to deflection in a first plane and the second active deflection section is limited to deflection in a second different plane. The first plane is angled to the second plane.

### BACKGROUND OF THE INVENTION

1. Field of the Invention

The present invention relates to medical instruments and, more particularly, to an endoscope.

2. Brief Description of Prior Developments

U.S. Pat. No. 4,873,965 discloses a flexible endoscope with two articulated lengths. U.K. patent application No. 2130885 discloses a flexible distal end portion for an endoscope. The end portion is made from plastic material with vertebrae connected by an elongate member or spine. U.S. Pat. No. 5,938,588 discloses an endoscope with wire sheaths made as solid tubes from a superelastic alloy material. Endoscopes are also known in the art which comprise an active deflection section and a passive deflection section.

### SUMMARY OF THE INVENTION

In accordance with one aspect of the present invention, an endoscope is provided including a handle; and a shaft extending from the handle. The shaft has a front end with a first active deflection section and a second active deflection section. The first active deflection section is limited to deflection in a first plane and the second active deflection section is limited to deflection in a second different plane. The first plane is angled to the second plane.

In accordance with another aspect of the present invention, a nephroscope is provided adapted to be inserted through an incision in a renal pelvis of a patient. The nephroscope comprises a handle having a control section; and a shaft extending from the handle. The shaft comprises a front end with a first active deflection section connected in series with a second active deflection section. The control section is adapted to independently deflect the first and second deflection sections. The first and second active deflection sections are adapted to deflect such that a distal end of the nephroscope can be placed in a calyx of a lower pole of a kidney without the need to passively deflect the front end of the shaft against tissue of the kidney of a patient to reach the calyx of the lower pole. The first and second active deflection sections are each limited to deflection in a single common plane relative to each other.

In accordance with one method of the present invention, a method is provided for viewing an area inside a patient with an endoscope. The method comprises steps of a) moving a second user actuated control of the endoscope to move a second active deflection section at a front end of a shaft of the endoscope, the second active deflection section being limited to movement along a single plane, the step of moving the second user actuated control moving a distal tip of the shaft of the endoscope along a first path limited to the plane without moving a first user actuated control of the endoscope; b) moving the first user actuated control to move a first active deflection section at the front end of the shaft to move the distal tip in a second path orthogonal to the first path without moving the second user actuated control; and c) repeating steps a) and b) for methodically scanning the area inside the patient by a series of adjacent parallel ones of the first paths.

### BRIEF DESCRIPTION OF THE DRAWINGS

The foregoing aspects and other features of the present invention are explained in the following description, taken in connection with the accompanying drawings, wherein:

FIG. 1 is a side elevational view of an endoscope incorporating features of the present invention;

FIG. 2 is an enlarged perspective view of the front end of the endoscope shown in FIG. 1;

FIG. 3 is a side elevational view of the front end of the endoscope shown in FIG. 1 with the outer cover removed;

FIG. 3A is a cross sectional view taken along line 3A-3A of FIG. 3;

FIG. 3B is a cross sectional view taken along line 3B-3B of FIG. 3;

FIG. 3C is a cross sectional view taken along line 3C-3C of FIG. 3;

FIG. 3D is a cross sectional view taken along line 3D-3D of FIG. 3;

FIG. 3E is an enlarged partial cross sectional view of area 3E shown in FIG. 3;

FIG. 3F is an enlarged partial cross sectional view of area 3F shown in FIG. 3;

FIG. 4A is an end view of the ring member shown in FIG. 3B;

FIG. 4B is a cross sectional view of the ring member shown in FIG. 4A taken along line 4B-4B;

FIG. 4C is a side elevational view of the ring member shown in FIG. 4A;

FIG. 5A is an end view of the ring member shown in FIG. 3D;

FIG. 5B is a cross sectional view of the ring member shown in FIG. 5A taken along line 5B-5B;

FIG. 5C is a top plan view of the ring member shown in FIG. 5A;

FIG. 6A is a diagrammatic view of the front end of the endoscope shown in FIG. 1 with the first and second active deflection sections in straight positions;

FIG. 6B is a diagrammatic view as in FIG. 6A with the first active deflection section bent upward;

FIG. 6C is a diagrammatic view as in FIG. 6B with the second active deflection section sent to a left side;

FIG. 6D is a diagrammatic view as in FIG. 6C with the first active deflection section bent in a downward position;

FIG. 6E is a diagrammatic view as in FIG. 6D with the second active deflection section bent to a right side;

FIG. 6F is a diagrammatic view as in FIG. 6E with the first active deflection section bent in an upward direction;

FIG. 7A is a diagrammatic cross sectional view of the front end of the endoscope shown in FIG. 1 located inside a bladder of the patient with the front end of the staff in the position shown in FIG. 6C;

FIG. 7B is a diagrammatic cross sectional view as in FIG. 7A with the front end of the shaft in the position shown in FIG. 6D;

FIG. 8A is a diagrammatic cross sectional view of the front end of the endoscope shown in FIG. 1 located inside the bladder of the patient as shown in FIG. 7A;

FIG. 8B is a diagrammatic cross sectional view as in FIG. 8A with the front end of the endoscope in the position as shown in FIG. 7B;

FIG. 9 is a cross sectional view of a bladder;

FIG. 10 is a cross sectional view of a kidney having the front end of a nephroscope similar to the endoscope shown in FIG. 1 located therein;

FIG. 11 is side elevational view of frame members and a fitting used to form part of the front end of the nephroscope shown in FIG. 10; and

FIG. 12 is a schematic view of portions of frame sections and control wires of an alternate construction of the first and second active deflection sections incorporating features of the present invention.

### DETAILED DESCRIPTION OF THE PREFERRED EMBODIMENTS

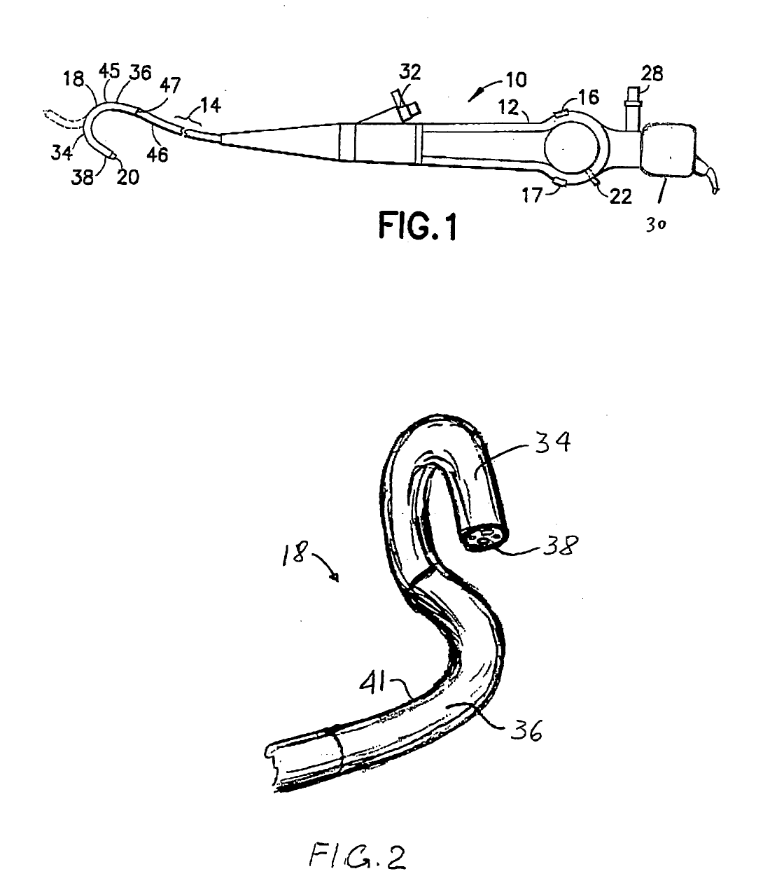

Referring to FIG. 1, there is shown a side elevational view of an endoscope 10 incorporating features of the present invention. Although the present invention will be described with reference to the embodiments shown in the drawings, it should be understood that the present invention can be embodied in many alternate forms of embodiments. Features of the present invention can be embodied in various different types of flexible, deflectable endoscopes. In addition, any suitable size, shape or type of elements or materials could be used.

The endoscope 10, in this embodiment, is a flexible cystoscope. However, in alternate embodiments, the endoscope could be a nephroscope, a cysto-nephroscope, or any other suitable type of endoscope. The endoscope 10 generally comprises a handle 12, a flexible shaft 14 connected to the handle 12, and a front end 18 of the shaft which has an active deflection capability. In an alternate embodiment, the shaft 14 could be rigid except at its front end.

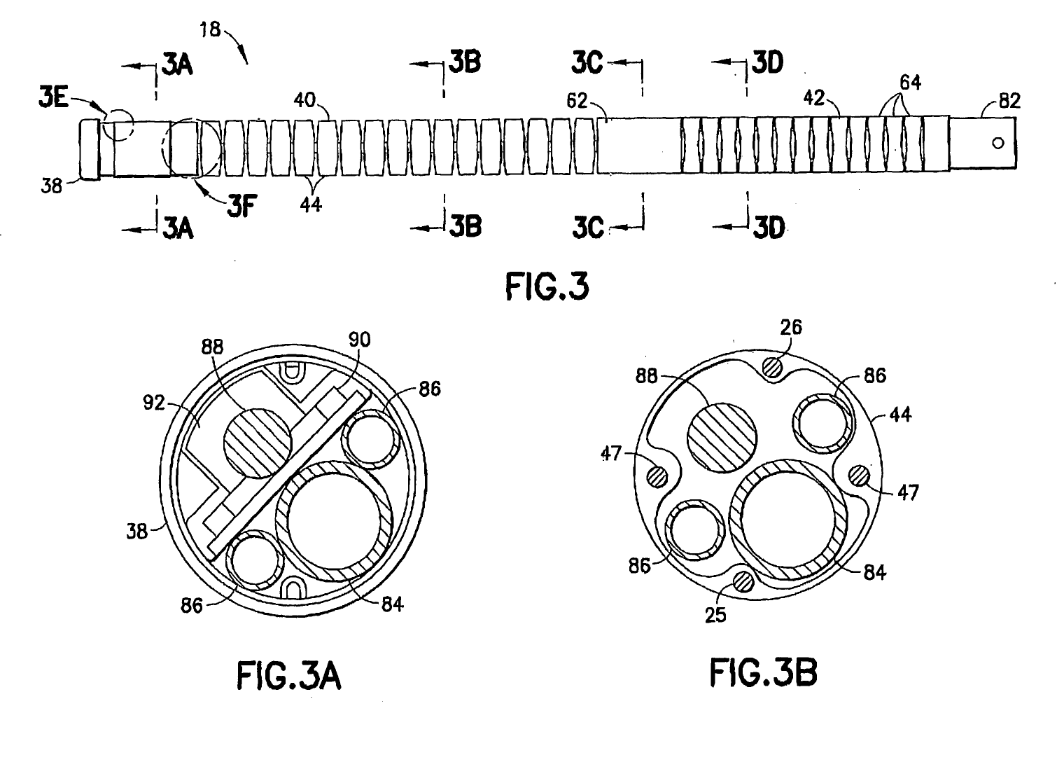

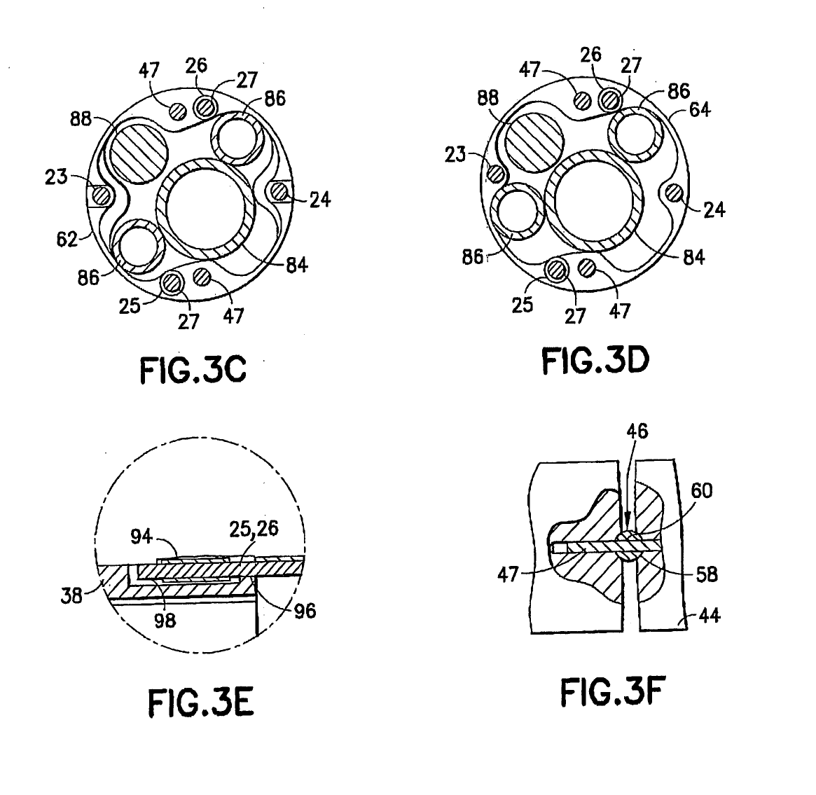

The handle 12 is part of a control system to control the active deflection capability of the front end 18. The control system generally comprises the handle 12, two actuators 16, 17, a brake or lock actuator 22, and four control wires 23, 24, 25, 26 (see FIGS. 3A-3F). However, in alternate embodiments, the control system could comprise additional or alternative components.

The three actuators 16, 17, 22 are movably attached to the handle 12. Proximal ends of the wires 23, 24, 25, 26 are connected to the two control actuators 16, 17. The brake actuator 22 is connected to a braking mechanism for locking the second control actuator 17 at a fixed position. However, in an alternate embodiment, any suitable type of brake or locking mechanism could be provided. In one type of alternate embodiment, the endoscope might not comprise a control actuator brake.

In the embodiment shown, the first control actuator 16 does not comprise a brake. In an alternate embodiment the endoscope could comprise two brakes. However, a brake could be provided for the first control actuator 16. In an alternate embodiment, the control could comprise a joystick type of control device.

The handle 12 also comprises a light source post 28, a connection section 30 for connecting the output to a display devise (not shown), and working instrument/irrigation inlets 32. However, in alternate embodiments, the handle 12 could comprise additional or alternative components. The instrument includes two fiber-optic illumination bundles 86 which extends through the shaft 14 between the light post 28 and the distal end 20. In an alternate embodiment, a fiber optic image bundle could extend through the shaft 14 between the distal end 20 and an eyepiece (not shown) which would replace the output connection section 30. A working channel 84 extends through the shaft 14 between the working instrument inlet 32 and the distal end 20.

The flexible shaft 14 behind the front end 18 could comprise any suitable type of flexible shaft, such as the shaft disclosed in U.S. patent application Ser. No. 09/547,686 which is hereby incorporated by reference in its entirety. The front end 18 comprises a first active deflection section 34 and a second active deflection section 36. Referring also to FIG. 2, in this embodiment the second active deflection section 36 is limited to deflection in a single plane relative to the handle 12 and the first active deflection section 34 is limited to deflection in a single different plane relative to the second active deflection section 36. In particular, the two planes are substantially orthogonal to each other such as at an angle of about 75° to about 85°, but could be as much as 90°. The second active deflection section 36 can bend in right and left directions and the first active deflection section 34 can bend in upward and downward directions. In alternate embodiments, the first and/or second active deflection sections could each be more or less than two way deflectable.

Referring also to FIGS. 3-5, the front end 18 generally comprises a distal end member 38, two frames 40, 42 connected to each other by a fitting or coupling 62, and an outer cover 41 (see FIG. 2). In the embodiment shown, the two frames 40, 42 are similar to each other, but are orientated about 75° to about 85° axially rotated relative to each other at the fitting 62. In alternate embodiments the two frames 40, 42 could be different from one another.

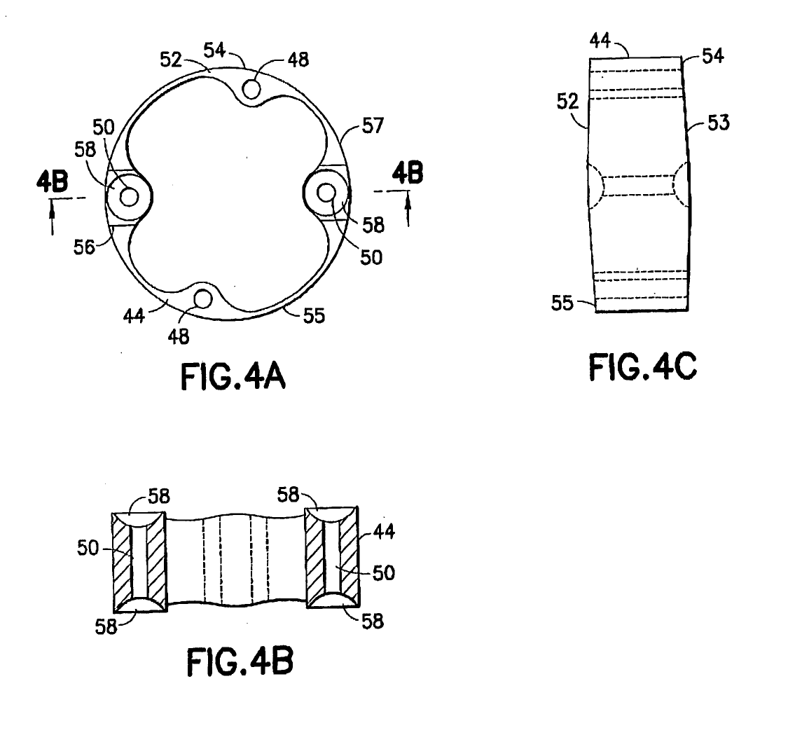

The first frame 40 generally comprises ring members 44, pivot bearings 46 (see FIG. 3F), and connecting members 47 (see FIG. 3F). Referring also to FIGS. 4A-4C, each ring member 44 is preferably comprised of stainless steel. However, any suitable material could be used. Each ring member 44 comprises a generally open center channel, two control wire channels 48 and two connecting member channels 50. The front side 52 and the rear side 53 of each ring member 44 is sloped inwardly such that widths of the lateral sides of the ring member are larger than the top and bottom of the ring member. The two control wire channels 48 extend through the top and bottom sides 54, 55. The two connecting member channels 50 extend through the lateral sides 56, 57. The front and rear sides of the connecting member channels 50 comprise sockets 58.

As seen best in FIG. 3F, each pivot bearing 46 is generally ball shaped with a center channel 60 therethrough. The ring members 44 are aligned in a series with two of the pivot bearings 46 located between each adjacent pair of the ring members 44. The pivot bearings 46 are located in the sockets 58. The pivot bearings 46, and the sloped shapes of the front and rear sides 52, 53 of the ring members 44, allow the ring members 44 to pivot relative to each other in up and down directions.

The connecting members 47 extend through the connecting member channels 50 and channels 60 in the pivot bearings 46. The connecting members 47 can comprise a wire or cable. In a preferred embodiment, the first frame 40 comprises two of the connecting members 47; one through the left side and one through the right side. The ends of the connecting members 47 extend into the coupling 62 and the distal end member 38. In a preferred embodiment, the opposite ends of the connecting members 47 are free to move relative to the coupling 62 and distal end member 38. However, in an alternate embodiment, one of the opposite ends of the connecting members 47 could be fixedly attached.

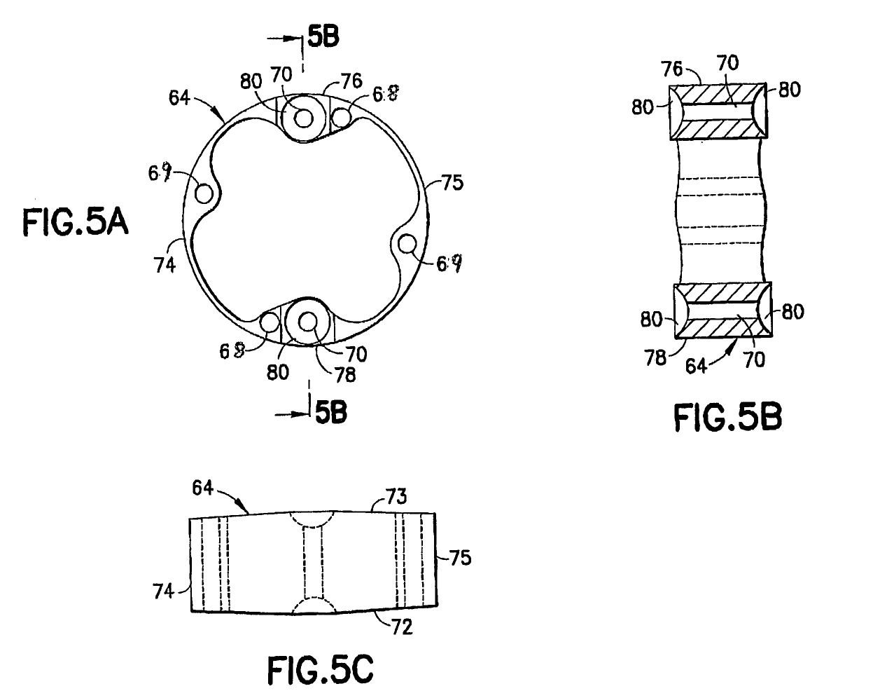

The second frame 42 generally comprises ring members 64, pivot bearings 46 (see FIG. 3F), and connecting members 47 (see FIG. 3F). Referring also to FIGS. 5A-5C, each ring member 64 is preferably comprised of stainless steel. However, any suitable material could be used. Each ring member 64 comprises a generally open center channel, a first set of control wire channels 68, a second set of control wire channels 69, and two connecting member channels 70. The front side 72 and the rear side 73 of each ring member 64 is sloped inwardly such that widths of the top and bottom sides of the ring member are larger than the middle section of the ring member. The two control wire channels 68 of the first set of control wire channels extend through the top and bottom sides 76, 78. The two control wire channels 69 of the second set of control wire channels extend through the lateral sides 74, 75. The two connecting member channels 70 extend through the top and bottom sides 76, 78. The front and rear sides of the connecting member channels 70 comprise sockets 80.

The pivot bearings used in the second frame 42 are the same as the pivot bearings used in the first frame 40. Similar to that seen in FIG. 3F, each pivot bearing 46 is generally ball shaped with a center channel 60. The ring members 64 are aligned in a series with two of the pivot bearings 46 located between each adjacent pair of the ring members 64. The pivot bearings 46 are located in the sockets 80. The pivot bearings 46, and the sloped shapes of the front and rear sides 72, 73 of the ring members 64, allow the ring members 64 to pivot relative to each other in right and left directions.

The connecting members used in the second frame 42 are identical to the connecting members 47 used in the first frame 40 (see FIG. 3F). The second frame 42 comprises two of the connecting members 47. The connecting members 47 extend through the connecting member channels 70 and channels 60 in the pivot bearings 46. The connecting members 47 can comprise a wire or cable. In a preferred embodiment, the second frame 42 comprises two of the connecting members 47; one through the top side and one through the bottom side. The ends of the connecting members 47 extend into the coupling 62 and proximal end member 82. In a preferred embodiment, the opposite ends of the connecting members 47 are free to move relative to the coupling 62 and proximal end member 82. However, in an alternate embodiment, one of the opposite ends of the connecting members 47 could be fixedly attached.

Referring particularly to FIGS. 3A, 3B, 3C and 3D, the shaft 14 comprises various components passing through it including a working channel 84, two light carriers 86, and an electrical cable 88. These four members extend through the center channels of the various ring members 44, 64 from the handle 12 up to the distal end 20. As shown best in FIG. 3A, the electrical cable 88 is connected to a printed circuit board 90 inside the distal end member 38. An image capturing system 92 is attached to a sensor on the printed circuit board 90 and has the electrical cable 88 connected thereto. The user can view the image from the image capturing system 92 at the at a video display (not shown) if the cable 88 is connected to such a display. In an alternate embodiment, the electrical cable 88, printed circuit board 90 and image capturing system 92 could be replaced by a fiber optic image bundle and objective lens. In this alternate embodiment the user could view the image at an eyepiece at the end of the handle 12, or with a snap-on camera.

Distal ends of a first set of two of the control wires 25, 26 are connected to the distal end member 38. Referring to FIG. 3E, the connection of one of the control wires 25, 26 to the distal end member 38 will be described. The control wires 25, 26 are inserted through holes 96 at the rear end of the distal end member 38. A sleeve 94 is fixedly attached to each of the distal ends of the control wires 25, 26. The control wires 25, 26 are then pulled to taunt and the distal end of the wires and sleeve 94 are located in pockets 98 of the distal end member 38. Thus, when the control wires 25, 26 are pulled rearward, they can pull on the distal end member 38 in a rearward direction. One of the control wires 25, 26 is pulled rearward by the actuators 16 while the other control wire is released by the actuator. Thus, the first frame 40 can bend up or down. However, because of the couplings provided among the ring members 44 at the pivot bearings 46, the movement of the frame 40 is limited to only two directions in a single plane.

Referring to FIGS. 3C and 3D, the ring members 64 and the coupling 62 comprise channels which allow the first set of control wires 25, 26 to pass therethrough with outer cable sheaths 27. Distal ends of two of the second set of control wires 23, 24 are connected to the coupling 62. However, the control wires 23, 24 for the second active deflection section 36 are offset relative to the control wires 25, 26 for the first active deflection section 34 about 90 degrees (such as about 75° to about 85°). The connection of the second control wires 23, 24 to the coupling 62 is substantially the same as the connection of the first control wires 25, 26 to the distal end member 38 (as seen in FIG. 3E; namely, by use of sleeves 94 which are located in receiving pockets of the coupling 62). Thus, when the second control wires 23, 24 are pulled rearward, they can pull on the coupling 62 in a rearward direction. One of the control wires 23, 24 is pulled rearward by the actuator 16 while the other control wire is released by the actuator. Thus, the second frame 42 can bend left or right. However, because of the couplings provided among the ring members 64 at the pivot bearings 46, the movement of the frame 42 is limited to only two directions in a single plane.

In the cross sectional views shown in FIGS. 3B, 3C and 3D, the first control cables 25, 26 and the connecting members 47 in the second frame 42 are slightly offset from each other. Thus, the two planes of deflection provided by the two frames 40, 42 are not precisely orthogonal to each other. However, in an alternate embodiment, the connecting members 47 in the rear frame 42 could comprise a general tube shape with the control wires 25, 26 extending therethrough. Thus, a truly orthogonal arrangement could be provided.

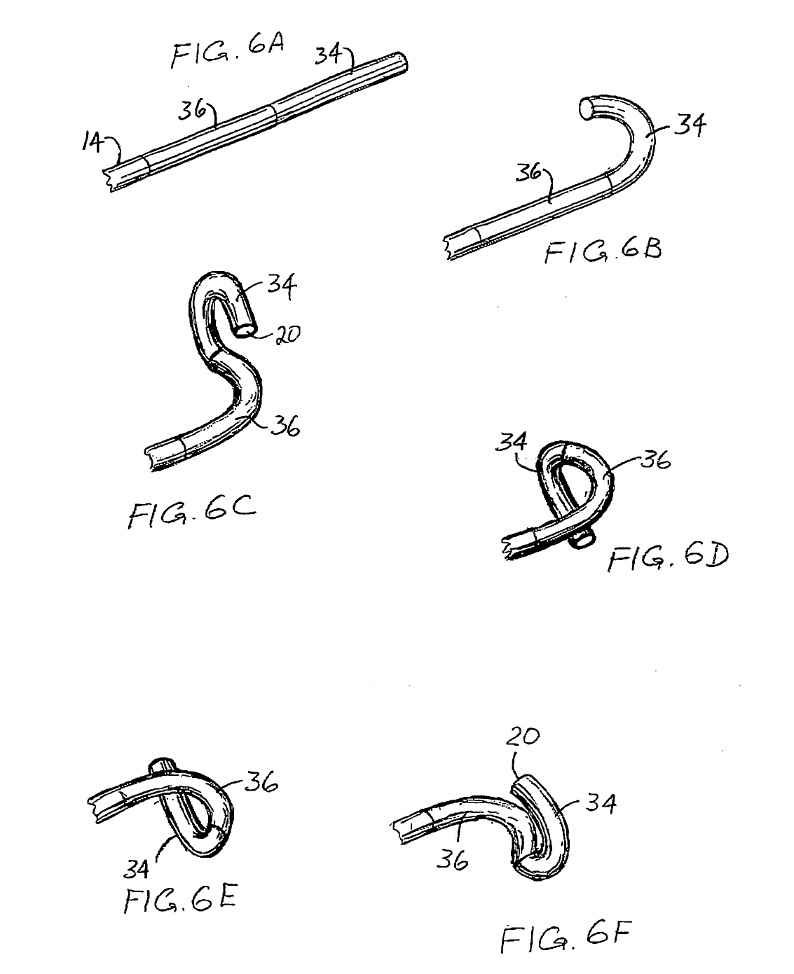

Referring now also to FIGS. 6A-6F, movements of the first and second active deflections sections 34, 36 will be described. FIG. 6A shows the first and second active deflections sections 34, 36 in substantially straight orientations relative to each other. This type of configuration is used to insert the front end of the endoscope 10 into the interior of a patient bladder 100 through the patient's urethra 102 (see FIG. 9). Once inserted into the interior of the bladder 100, the user can then manipulate the actuators 16, 17 to separately and independently bend the first and second active deflections sections 34, 36.

FIG. 6B shows the first active deflection section 34 being bent upward while the second active deflection section 36 is maintained in a straight direction. FIG. 6C shows the first active deflection section 34 being maintained in its upwardly bent position while the second active deflection section 36 has been bent to the left.

As can be seen, the distal end 20 of the endoscope 10 faces an upward and rearward direction. FIG. 6D shows the second active deflection section 36 being maintained in its left bent position while the first active deflection section 34 has been bent from its upward bent position to its downward position. The distal end 20 of the endoscope 10 faces a downward and forward direction. FIG. 6E shows the first active deflection section 34 being maintained in its downward bent position while the second active deflection section 36 has been bent from its left side bent position to its right side bent position. Thus, the distal end 20 of the endoscope 10 faces a downward and rearward direction. FIG. 6F shows the second active deflection section 36 being maintained in its right side bent position while the first active deflections section 34 is bent back to its upward bent position. Thus, the distal end 20 of the endoscope faces an upward and a rearward direction.

As noted above, in this embodiment the first active deflection section 34 and the second active deflection section 36 are limited to deflection in single planes of deflection which are generally orthogonal to each other, such as generally horizontal and generally vertical. Referring also to FIG. 9, a cross sectional view of a bladder is shown. In this embodiment the endoscope is a cystoscope intended to be inserted into the bladder 100 via a urethra 102. Once front end 18 of the endoscope is inserted into the bladder 100, the front end 18 can be actively deflected to view the interior of the bladder, and perhaps perform operations inside the bladder.

The interior of the bladder has a general round, oval or global shape. Thus, it is necessary for the user of the endoscope to manipulate the endoscope to view an interior global 360° area. In the past, cystoscopes were provided with omni-directional controls that allowed for their single front end active deflection section to be four way deflectable. However, an attempt to systematically view all of the interior global area inside the bladder by merely using the controls (so that the user was certain that no portion of the area was missed) was virtually impossible because of the complexity and memory that would need to be used. Therefore, users in the past would often scan the interior global area in a systematic pattern by manually axially rotated the entire shaft of the endoscope relative to the urethra 102 and only actively deflecting the front end in one plane. However, because the cystoscope was rotated after each pass of scanning, the user needed to contort his body during the process.

The present invention allows a user to perform a systematic scanning process, but without the need to axially rotate the shaft of the cystoscope relative to the urethra, and without the user having to contort his body during the process. The rear end of the second active deflection section 36 can remain substantially stationary, but the construction of the two active deflection sections 34, 36 and the field of view of the image viewing system at the distal tip 20, still allows a 360° revolute viewing inside a general sphere shape (i.e., inside a bladder). The present invention accomplishes this ability by providing the front end of the cystoscope with two independently movable active deflection sections which are limited to single planes of deflection that are angled or generally orthogonal to each other. Thus, the front tip of the distal end member 38 can be moved in a first plane, such as horizontal, without moving it vertically to perform a first scanning path. The front tip of the distal end member 38 can be moved in a second plane, such as vertically, without moving it horizontally. The front tip can then be moved in a third plane, generally adjacent to the first scanning path, without moving it vertically, to perform a second scanning path adjacent to the first scanning path.

By physically limiting the front end to two orthogonal or angled independent single plane deflectable motions, the user can control the controls 16, 17 very easily without getting confused or, without the distal end member 38 moving in an unintended direction. For example, the user merely moves the control 16 to move the distal end member 38 upwards and downwards and does not have to move the control 17. Then, the user merely moves the control 17 to move the distal end member 38 right and left and does not have to move the control 16. In the prior art, the user had to move both controls at the same time to obtain this single plane type of movement with repeated consistency. Moving both controls at the same time to obtain this single plane type of movement with repeated consistency was just too complicated and time consuming and, thus, users merely reverted to the rotation of shaft relative to the patient as described above.

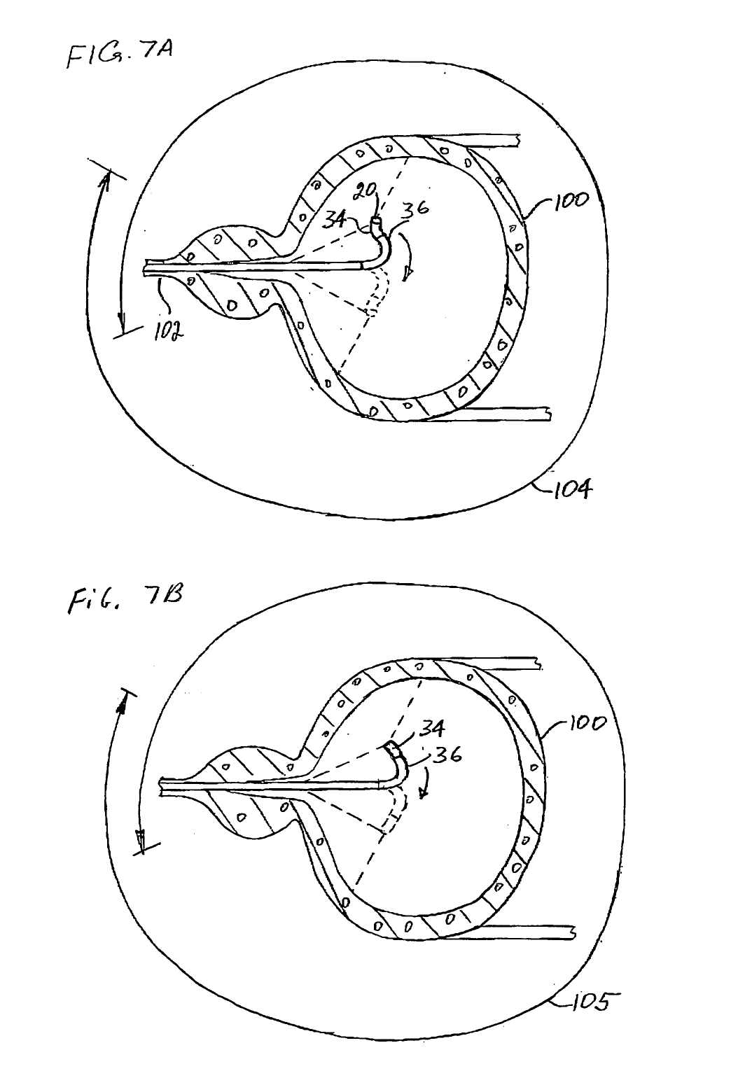

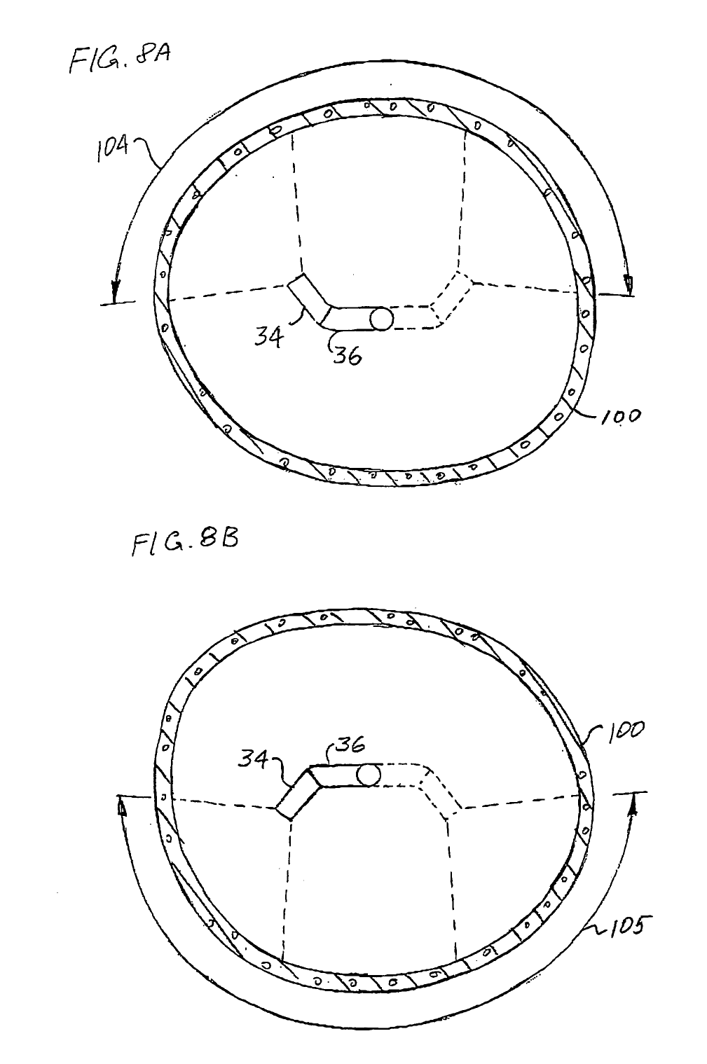

One of the unique features of the present invention is the ability to allow the user to controllably view an interior surface of a patient in a controlled and methodical manner. More specifically, referring also to FIGS. 7A, 7B, 8A and 8B, the front end of the endoscope 10 is shown inside a bladder 100. The user can initially use the actuators 16, 17 to position the distal end 20 as shown in FIG. 7A with the second active deflection section 36 bent to the left side and the first active deflection section 34 bent in an upward direction. The user can then manipulate only one of the actuators 17 to deflect the second active deflection section 36 from its left side bent shape to its right side bent shape as illustrated by the phantom lines shown in FIG. 7A. The other actuator 16 is not moved by the user and, therefore, the first active deflection section 34 is retained in its upward bent shape throughout the entire motion of the second active deflection section 36 moving from its left bent position to its right bent position. The camera at the distal end 20 is able to scan the interior of the bladder 100 along a path 104. Referring also to FIG. 8A, in a preferred embodiment the path 104 is about 50 percent of the entire interior surface of the bladder 100; the upper half of the bladder's interior surface.

The user can then returned the second active deflection section 36 back to its left bent position shown in FIG. 7A. By manipulating only the first actuator 16, the user can then deflect the first active deflection section 34 from its upward bent position to its downward bent position as seen in FIGS. 7B and 8B. The user can then manipulate merely the second actuator 17 to deflect the second active deflection section 36 from its left side bent to its right side bent as illustrated by the phantom lines shown in FIG. 7B. The camera at the distal end 20 is able to scan the interior of the bladder 100 along a path 105. In a preferred embodiment, the path 105 is about 50 percent of the entire interior surface of the bladder 100; the lower half. However, in alternate embodiments, the scanning paths may comprise more than the two paths 104, 105 and, but the scanning paths are preferably adjacent or partially overlap each other. In an alternate method, the user could scanned by moving the first active deflection section 34 and keeping the second active deflection section 36 stationary.

With the present invention, a user can systematically scan adjacent paths to view the entire revolute 360° area inside the bladder. The method can comprise moving a second user actuated control of the endoscope to move a second active deflection section at a front end of a shaft of the endoscope, the second active deflection section being limited to movement along a single plane, the step of moving the second user actuated control moving a distal tip of the shaft of the endoscope along a first path limited to the plane without moving a first user actuated control of the endoscope; moving the first user actuated control to move a first active deflection section at the front end of the shaft to move the distal tip in a second path generally orthogonal to the first path without moving the second user actuated control; and repeating these two steps for methodically scanning the area inside the patient by a series of adjacent ones of the first paths.

In a preferred embodiment, the first active deflection section is adapted to deflect through an angle of about 110° to about 210° and, the second active deflection section is adapted to deflect through an angle of about 110° to about 210°; and preferably about 130° each. However, any suitable angles could be provided. The field of view of the optical lens at the front tip of the endoscope allows viewing 360° when moved through these angles. The endoscope preferably only comprises one brake for one of the controls, such as only for the left and right control. However, in an alternate embodiment the one brake might control only up and down braking control. The present invention forms a means for viewing an inside of a generally spherical shape through a fixed entrance into the generally spherical shape by a camera or an optical lens at the front end of the shaft without axially rotating the shaft.

The present invention provided an advantage of allowing an interior global scanning without substantially any shaft rotation needed. With the present invention of generally orthogonal, serially connected two-way only active deflection sections, the user has better control over movement of the distal tip (and thus the path(s) being viewed). The user can, thus, use a controlled systematic and methodical scanning pattern method to add certainty that an entire interior global area has been observed. The user can scan a path in merely one direction and reposition in an orthogonal direction to subsequently take another adjacent scan path. Thus, a scan-reposition repetition method can be used which can allow a user to limit the two step method to movement of a single one of the controls 16, 17 for each respective step. This provides a clearly defined scanning pattern and stepped movement of the controls 16, 17 for stepped movement of the sections 34, 36.

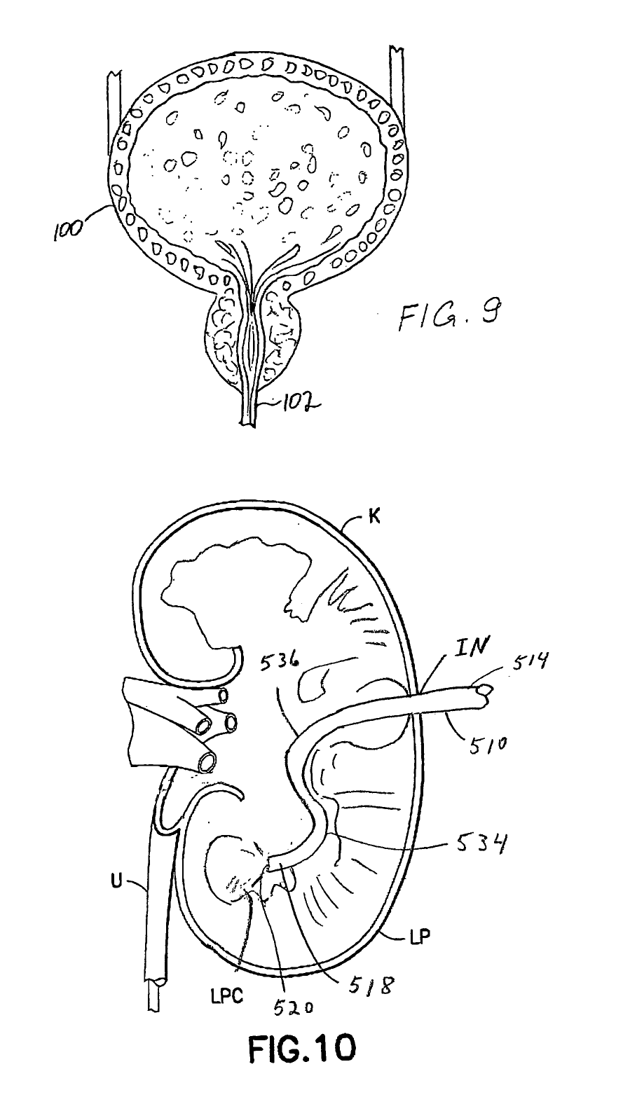

Referring now to FIG. 10, a cross sectional view of a kidney K is shown with the front end 518 of an alternate embodiment of the present invention located therein. In this embodiment the endoscope 510 is a nephroscope (an instrument inserted into an incision IN in the renal pelvis for viewing the inside of the kidney) or a cysto-nephroscope which can be used both as a cystoscope or a nephroscope. The two active deflection sections 534, 536 are adapted to allow the distal end 520 of the endoscope to project into a calyx LPC of the kidney in the lower lobe or lower pole LP. More specifically, the two active deflection sections 534, 536 are adapted to allow the distal end 520 to project into the calyx LPC in the lower lobe LP without passively deflecting the front end 518 off of kidney tissue of the patient. The front end 518 does not comprise a passive deflection section. Instead, the front end 518 comprises the two active deflection sections as described herein.

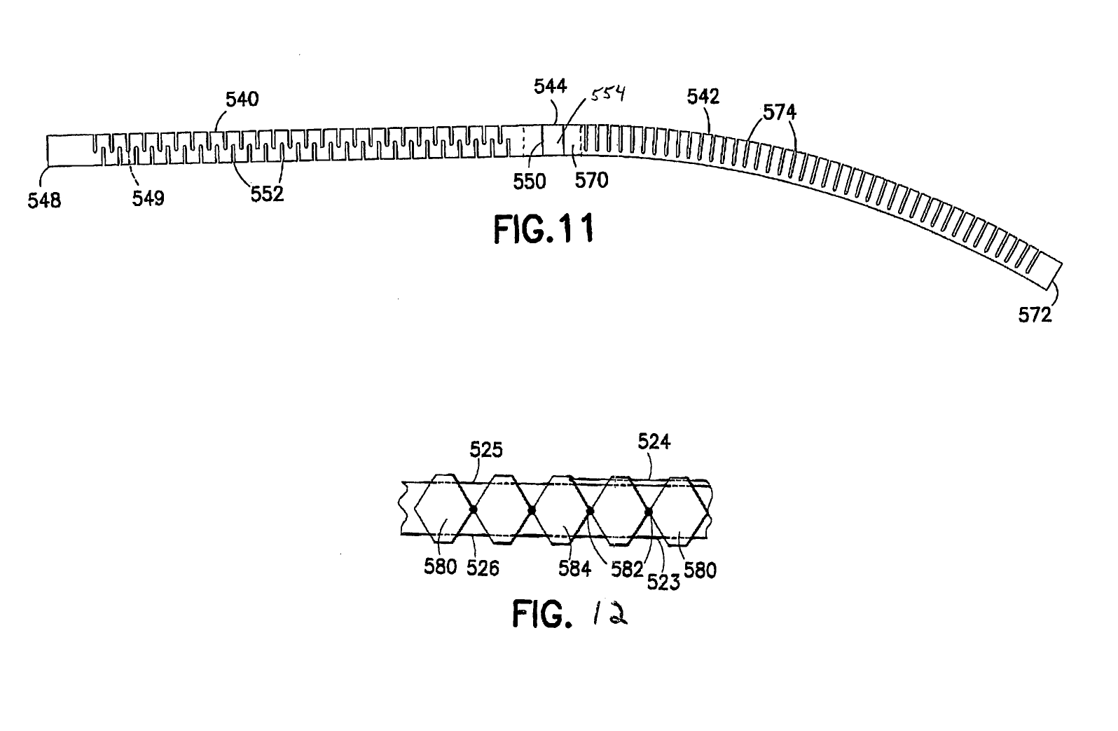

Referring also to FIG. 11, components of the front end 518 will be described. The front end 518 generally comprises a distal end member, a first frame member 540, a second frame member 542 and a fitting 544. The front end 518 also comprises an elastomeric cover which is attached at a sealed joint to a cover which extends the entire length of the shaft 514. The distal end member is connected to a front end of the first frame member 540. The front ends of two first control wires are fixedly attached to the distal end member.

The first frame member 540 generally comprises a single one-piece generally tubular shaped member. However, in alternate embodiments, the first frame member 540 could be comprised of more than one tube, such as multiple tubes connected in series, and could comprise additional members. The first frame member 540 is preferably comprised of a shape memory alloy material, such as Tinel or Nitinol. However, any suitable type of shape memory alloy material could be used. The shape memory alloy material is used for its superelastic properties exhibited by the material's ability to deflect and resiliently return to its natural or predetermined position even when material strains approach 4%, or an order of magnitude greater than the typical yield strain of 0.4% giving rise to plastic deformation in common metals. Thus, the term “superelastic material” is used to denote this type of material.

The first frame member 540 has a center channel with open front and rear ends 548, 550, and slots 552 therein. The first frame member 540 forms the frame for the first active deflection section 534. The slots 552, in the embodiment shown, extend into the first frame member 540 in two opposite directions. However, in alternate embodiments, the slots 552 could extend into the first frame member in more or less than two directions. The slots 552 extend into the first frame member 540 along a majority of the length of the first frame member, and also extend into the first frame member a distance more than half the diameter. However, in alternate embodiments, the slots 552 could be arranged in any suitable type of array or shape.

The rear end 550 of the first frame member 540 is fixedly attached to the fitting 544. The fitting 544 is comprised of a one-piece member made of a suitable material, such as metal. However, in alternate embodiments, the fitting 544 could be comprised of more than one member, or could be incorporated into one or both of the frame members, and could be comprised of any suitable type of material(s). The rear end 550 of the first frame member 540 is fixedly attached to the exterior of the front of the fitting 544.

The center section 554 forms a raised annular ring around the fitting 544. This raised annular ring forms stop surfaces for the ends 550, 570 of the two frame members. In alternate embodiments, any suitable type of positioning system for positioning the frame members on the fitting could be provided.

The inside of the fitting 544 generally comprises two pass-through holes and a mounting section for mounting an end of a second control cable thereto. The two pass-through holes are sized and shaped to allow the two first control cables to slidably pass therethrough. The fitting 544 has a mounting section which comprises an aperture that is sized and shaped to receive the front end of the second control cable 524, such that the front end can the fixedly mounted therein. However, in alternate embodiments, any suitable means could be used to attach the front end of the second control cable to the fitting 544. In addition, in an alternate embodiment, the fitting 544 could be adapted to have more than one control cable fixedly mounted thereto. In addition, in another alternate embodiment, the fitting 544 could be adapted to have more or less than two control cables pass therethrough.

The second frame member 542 generally comprises a single one-piece generally tubular shaped member. However, in alternate embodiments, the second frame member 542 could be comprised of more than one tube, such as multiple tubes connected in series, and could comprise additional members. The second frame member 542 could also be comprised of a front portion of a member which extends along the length of the shaft 514, similar to that disclosed in U.S. patent application Ser. No. 09/427,164. The second frame member 542 is preferably comprised of a shape memory alloy material, similar to that described above with reference to the first frame member 540.

The second frame member 542 has a center channel with open front and rear ends 570, 572, and slots 574 therein. The second frame member 542 forms the frame for the second active deflection section 536. The slots 574, in the embodiment shown, extend into the second frame member 542 in only one direction. However, in alternate embodiments, the slots 574 could extend into the second frame member in more than one direction. The slots 574 extend into the second frame member 542 along a majority of the length of the second frame member, and also extend into the second frame member a distance of about three-quarters of the diameter. However, in alternate embodiments, the slots 574 could be arranged in any suitable type of array, or shape, or depth of extension into the lateral side of the frame member.

In the embodiment shown, the second frame member 542 comprises a curved pre-shaped home position as shown in FIG. 11. However, in an alternate embodiment, the second frame member 542 could comprise any suitable type of pre-shaped home position, including a straight home position similar to the first frame member 540. In an alternate embodiment, the first frame member 540 could comprise a curved pre-shaped home position. The front end 570 of the second frame member 542 is fixedly attached to the rear section of the fitting 544. The rear end 572 of the second frame member 542 is fixedly attached to the frame of the shaft 514 located behind the front end 518.

In alternate embodiments, the first frame member 540 and/or the second frame member 542 could be comprised of any suitable material(s) and/or members. For example, the members could be comprised of metal rings connected by flexible members (such as rods of superelastic material or other flexible material), or could merely comprise metal rings pivotably connected to each other. Features of the present invention are not necessarily limited to use of only two tube shaped frame members comprised only of superelastic material. For example, one type of alternate embodiment is shown in FIG. 12. In this alternate embodiment the first and second active deflection sections comprise ring members 580. The ring members 580 are pivotably connected to each other in series at joints 582. First control wire 524 has its distal end connected to one of the ring members 584. The other control wires 525, 526 pass through the ring members. The metal rings could be comprised of stainless steel. Various different types of flexible, deflectable endoscope shaft constructions are known in the art which could be adapted or modified to practice the present invention. For example, the metal rings could be riveted to each other or hinged to each other to provide a pivotal movement.

In the embodiment shown, the lateral side of the second frame member 542, which the slots 574 extend into, is aligned with the mounting section of the fitting 544. The second control cable, when set by the actuator 17 to a predetermined position, applies tension to the fitting 544 such that the second frame member 542 is maintained in a substantially straight configuration. When the second control cable is pulled rearward by the actuator 17, the second frame member 542 is adapted to bend inward along the opposite lateral side. When the second control cable is released, internal stresses from the curved pre-shaped of the second frame member 542 cause the second active deflection section 536 to return to its home straight position. In an alternate embodiment, more or less than three control cables could be used.

Referring to FIG. 10 the control section (not shown) of the endoscope 510 is adapted to independently deflect the first and second active deflection sections. The first and second active deflection sections are adapted to deflect such that the distal end 520 of the nephroscope can be placed in the calyx LPC of the lower pole LP of the kidney K without passively deflecting the front end 518 of the shaft against tissue of the kidney to reach the calyx.

By providing the front end of the nephroscope with two active deflection sections, which are independently deflectable, the front end of the nephroscope is able to locate the distal end 520 in the calyx of a lower pole of a kidney regardless of the size or shape of the kidney. The nephroscope 510 is not dependent upon use of passive deflection against tissue of the kidney in order to properly position the distal end 520 at a desired position. The amount of space or real estate and the small radius turn into the calyx in the lower pole inside the kidney for manipulating the front end of the nephroscope 510 is very limited. The present invention, by using two separate shape memory frame members 540, 542 provides the ability to manipulate the front end 518 in this limited space and sharp turn path environment. The shape memory frame members 540, 542 provide superelastic properties to allow the frame members to deflect in this limited space and sharp turn path environment and be able to resiliently return to their home positions. The ability to independently deflect the two active deflection sections 534, 536 combined with the superelastic properties of the shape memory frame members allow the frame members to navigate a path through this limited space and sharp turn path environment. If only a single active deflection section was provided, it would be too long in length in order to operate properly to reach the calyx in the lower pole.

The first active deflection section 534 can be deflected before the second active deflection section 536, and the second active deflection section 536 can be deflected as it is enters the incision IN. This ability to provide a sequential deflecting of the active deflection sections 534, 536 as they exit the incision IN allows access to the lower pole calyx LPC without the use of passive deflection. The present invention provides the ability to reach previously unavailable areas in a kidney.

In alternate embodiments of the present invention, the front end 518 could comprise more than two active deflections sections. The first active deflection section 534 has been described above as being two-way deflectable in a same plane and in a same plane with the one way deflection of the second active deflection section 546. In another alternate embodiment, the first active deflection section 534 could be deflectable in more or less than two ways. In such an alternate embodiment, the control system could comprise more or less than two control cables for the first active deflection section. The second active deflection section 536 has been described above as being one way deflectable. In another alternate embodiment, the second active deflection section 536 could be deflectable in more than one way in a substantially same plane. In such an alternate embodiment, the control system could comprise more than one control cable for the second active deflection section.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

What is claimed is:

1. An endoscope comprising:
a handle; and
a shaft extending from the handle, the shaft having a front end comprising a first active deflection section including a plurality of rings pivotably connected to each other and a second active deflection section including a plurality of rings pivotably connected to each other, wherein the first active deflection section is limited to deflection in a first plane and the second active deflection section is limited to deflection in a second different plane, and wherein the first plane is angled to the second plane.

2. An endoscope as in claim 1 wherein the first plane is about 75° to about 90° relative to the second plane.

3. An endoscope as in claim 1 wherein the second active deflection section is limited to deflect only in left and right directions relative to the handle.

4. An endoscope as in claim 3 wherein the first active deflection section is limited to deflect only in upward and downward directions relative to the second active deflection section.

5. An endoscope as in claim 1 wherein the first active deflection section is adapted to deflect through an angle of about 110° to about 210°.

6. An endoscope as in claim 5 wherein the second active deflection section is adapted to deflect through an angle of about 110° to about 210°.

7. An endoscope as in claim 1 wherein the handle comprises a control section comprising a first actuator for moving only the first active deflection section and a second actuator for moving only the second active deflection section, and at least one brake actuator, the at least one brake actuator comprising a first brake actuator being adapted to lock only one of the active deflection section at a desired position.

8. An endoscope as in claim 7 wherein the at least one brake actuator comprises only one brake.

9. An endoscope as in claim 1 wherein the endoscope comprises a cystoscope comprising means for viewing 360° inside of a generally spherical shape through a fixed entrance into the generally spherical shape by a camera or an optical lens at the front end of the shaft without axially rotating the shaft.

10. An endoscope as in claim 1 wherein the first active deflection section comprises rings pivotably connected to each other to form a frame of the first active deflection section, wherein a connection of the rings to each other comprises balls located in sockets of the rings and at least one connecting member extending through a hole in the balls.

11. A nephroscope adapted to be inserted through an incision in a renal pelvis of a patient, the nephroscope comprising:
a handle having a control section; and
a shaft extending from the handle, the shaft comprising a front end with a first active deflection section connected in series with a second active deflection section, the control section being adapted to independently deflect the first and second deflection sections, wherein the first and second active deflection sections are adapted to deflect such that a distal end of the nephroscope can be placed in a calyx of a lower pole of a kidney without the need to passively deflecting the front end of the shaft against tissue of the kidney of a patient to reach the calyx of the lower pole, and wherein the first and second active deflection sections are each limited to deflection in a single common plane relative to each other.

12. A nephroscope as in claim 11 wherein the first active deflection section is adapted to deflect through an angle of about 180° to about 210°.

13. A nephroscope as in claim 11 wherein the control section comprises a first actuator for moving the first active deflection section and a second actuator for moving the second active deflection section, and at least one brake actuator, the at least one brake actuator comprising a first brake actuator being adapted to lock only the second active deflection section at a desired position.

14. A nephroscope as in claim 13 wherein the at least one brake actuator comprises only the first brake actuator.

15. A nephroscope as in claim 11 wherein the first active deflection section comprises a first shape-memory frame member having a general tubular shape comprised of superelastic material, and wherein the second active deflection section comprises a second shape-memory frame member having a general tubular shape comprised of superelastic material.

16. A nephroscope as in claim 15 wherein the second frame member has a curved pre-shaped home position.

17. A nephroscope as in claim 16 wherein the second frame member is maintained in a straight position by tension from a control wire from the control section.

18. A method of viewing an area inside a patient with an endoscope, the method comprising steps of:
a) moving a second user actuated control of the endoscope to move a second active deflection section at a front end of a shaft of the endoscope, the second active deflection section being limited to movement along a single plane, the step of moving the second user actuated control moving a distal tip of the shaft of the endoscope along a first path limited to the plane without moving a first user actuated control of the endoscope;
b) moving the first user actuated control to move a first active deflection section at the front end of the shaft to move the distal tip in a second path angled relative to the first path at an angle of about 73°-90° without moving the second user actuated control; and
c) repeating steps a) and b) for methodically scanning the area inside the patient by a series of adjacent parallel ones of the first paths.

19. A cysto-nephroscope comprising:
a handle;
a shaft extending from the handle, the shaft having a front end comprising a first active deflection section and a second active deflection section, wherein the first active deflection section is limited to deflection in a first plane and the second active deflection section is limited to deflection in a second different plane, wherein the first plane is angled to the second plane, and wherein the first active deflection section comprises rings which are each pivotably connected to an adjacent ring by two ball sections on opposite sides of each ring; and
means for viewing an inside of a generally spherical shape through a fixed entrance into the generally spherical shape by an optical lens at the front end of the shaft without axially rotating the shaft.

20. A cysto-nephroscope as in claim 19 further comprising a connecting member extending through holes in all of the ball sections on one of the sides.
